(19) United States
(12) Patent Application Publication
(10) Pub. No.: US 2006/0029625 A1
(43) Pub. Date: Feb. 9, 2006

(54) Personal cleansing composition containing fibers

(76) Inventor: Michael Frederick Niebauer, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/198,700

(22) Filed: Aug. 5, 2005

Related U.S. Application Data
(60) Provisional application No. 60/599,373, filed on Aug. 6, 2004.

(51) Int. Cl. A61K 8/02

(52) U.S. Cl. 424/401; 424/70.27

(57) ABSTRACT

Personal cleansing compositions comprise (a) from about 5 wt. % to about 50 wt. % of a water-soluble detersive surfactant; (b) at least about 0.1 wt. % of a fiber having a length and a thickness such that said length is greater than said thickness, wherein said length is from about 5 μm to about 1.5 mm; and (c) at least about 20 wt. % of an aqueous carrier; wherein said fiber forms a load-sensitive deposit upon dilution of said personal cleansing composition with water. Methods of providing both increased volume and superior styling and conditioning to hair comprise applying the personal care composition as described above to the hair and rinsing the hair.

### CROSS REFERENCE TO RELATED APPLICATIONS

This application claims the benefit of U.S. Provisional application Ser. No. 60/599,373 (Case 9738P), filed on Aug. 6, 2004.

### FIELD OF THE INVENTION

The present invention relates to personal cleansing compositions containing fibers. More specifically, it relates to personal cleansing compositions containing fibers which form a load-sensitive deposit. The compositions are intended to deliver the benefits of hair volume and fullness, as well as improved conditioning and combing.

### BACKGROUND OF THE INVENTION

Solid particles are known for use as benefit agents in a variety of formulations and personal care compositions. Solid particles can impart benefits both to the compositions comprising them or surfaces to which the compositions are applied. For examples, solid particles can be used as pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers. Additionally, many active ingredients useful as treatment agents for various disorders or socially embarrassing conditions are available and typically used in solid particulate form including antiperspirant agents, anti-dandruff agents, antimicrobials, antibiotics, and sunscreens.

Typically, when it is desired to modify the properties of a surface through application of particles, the particles are applied via leave-on preparations that are rubbed, sprayed, or otherwise applied directly onto the surface to be affected. Typical personal care preparations suitable for delivery of solid particles to hair or skin surfaces include moisturizers, lotions, creams, loose or pressed powders, sticks, tonics, gels, and various sprays such as aerosol or pump sprays. These products are usually applied directly to the surface whereupon particles are deposited and retained by the composition itself or by residual non-volatile elements of the composition after evaporation and drying.

It is also known to formulate solid particle benefit agents into rinse-off or cleansing compositions such as hair rinses, shampoos, liquid and bar soaps, conditioners, or colorants. Frequently, the solid particle benefit agent is used to affect the overall appearance, stability or aesthetics of the composition itself. For example, it is known to add colorant particles, pigments, or pearlescent agents to compositions to improve the acceptability and attractiveness of the product to potential consumers. It is also known to add particulate benefit agents to affect the in use performance, appearance or aesthetic properties of the composition or to provide a tactile signal to the user. For example, exfoliant particles are frequently used in cleansing compositions to improve abrasion and removal of oils and dirt from washed surfaces and to impart a perceptible “scrubbing” sensation to the user. Typically, such solid particle agents are not intended or desired to be deposited onto the substrate and are removed during dilution and rinsing of the composition from the surface to which they are applied.

It is also known to formulate solid particles into rinse-off or cleansing compositions to improve the volume, body, or fullness of the hair through increased interactions between hairs. When deposition of solid particle benefit agents from washing compositions is intended, the compositions available heretofore have suffered from the drawbacks of inefficient deposition, requiring the use of excess amounts of the particle agent or ineffective benefit delivery. Further, compositions containing solid particles intended to provide increased interaction between hairs frequently result in negative conditioning, wet detangling, and wet combing performance due to the frictional impact of the particles. Therefore, the negative conditioning and combing effects associated with the deposition of particles often outweigh the positive hair volume and body results. Prior attempts to mitigate the negative conditioning attributes of particle-containing compositions have either been insufficient or have negatively impacted the deposition or benefit associated with the particle.

In addition to particles, it is known to incorporate fibers into certain personal care and cosmetic compositions. Mascara compositions are known to incorporate fibers to provide lengthening effects to eyelashes. Other cosmetic compositions, such as skin make-up compositions, incorporate fibers to provide good feel and good cosmetic staying power to the compositions when applied to the skin. Such compositions, though, often suffer from stability and uniformity problems because the fibers are difficult to disperse homogeneously in compositions intended for such applications. Further, when applied as part of such compositions, the fibers sometimes flake off or detach from the hair or skin, or the fibers become tacky or sticky and clump together.

Fibers also have been incorporated into hair styling compositions, such as hair gels and hair sprays, to provide elasticity, volume, and hold benefits to hair. However, regular use of such compositions frequently results in build-up on the hair, which can negatively impact the feel and the style of the hair. Further, such compositions typically are applied as an additional step, separate from a shampooing and/or conditioning step, which increases the time and effort spent grooming.

Accordingly, a need still exists for a rinse-off personal cleansing composition which is capable of containing and effectively depositing a benefit agent on the treated surface to deliver improved hair volume, body, and fullness, while simultaneously delivering improved conditioning and combing performance. Moreover, a need still exists to provide such benefits from the deposition of fibers through the use of a rinse-off cleansing composition.

### SUMMARY OF THE INVENTION

It has now been discovered that the deposition of certain fibers onto the hair provides a load-sensitive deposit wherein, at very low force applied to the hair, the fibers act to increase hair friction which prevents hairs from moving past each other, and, at higher force applied to the hair, the hair friction is reduced to provide improved conditioning and combing performance relative to that of clean hair.

The present invention is directed to a personal cleansing composition comprising:
a) from about 5 wt. % to about 50 wt. % of a water-soluble detersive surfactant;
b) at least about 0.1 wt. % of a fiber having a length and a thickness such that said length is greater than said thickness, wherein said length is from about 5 μm to about 1.5 mm; and
c) at least about 20 wt. % of an aqueous carrier
wherein said fiber forms a load-sensitive deposit upon dilution of said personal cleansing composition with water.

The present invention is further directed to a method of using the personal cleansing composition.

These and other features, aspects, and advantages of the present invention will become evident to those skilled in the art from a reading of the present disclosure.

### DETAILED DESCRIPTION OF THE INVENTION

While the specification concludes with claims that particularly point out and distinctly claim the invention, it is believed the present invention will be better understood from the following description.

The personal cleansing compositions of the present invention include detersive surfactant, fibers, and an aqueous carrier. Each of these essential components, as well as preferred or optional components, are described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

The term “aspect ratio” as used herein refers to the ratio of the largest dimension of an object to the smallest dimension of an object.

The term “fiber” as used herein means a natural or synthetic object which is slender and elongated in shape, having a length and a thickness such that the length is greater than the thickness, and having a cross-section which is substantially circular, substantially elliptical, or substantially polygonal. The term “fiber” includes objects which are, for example, rod-like or ribbon-like in shape.

The term “load-sensitive deposit” as used herein refers to a material which, at very low force applied to the hair (e.g., the normal force present with hair-to-hair contact), acts to increase hair friction relative to the hair friction of clean hair, and, at higher force applied to the hair (e.g., the force present during combing), acts to decrease hair friction relative to the hair friction at very low force.

The term “polymer” as used herein shall include materials whether made by polymerization of one type of monomer or made by two (i.e., copolymers) or more types of monomers.

The term “water-soluble” as used herein means that a substance is soluble in water in the present composition. In general, the substance should be soluble at 25° C. at a concentration of at least about 0.1% by weight of the water solvent, preferably at least about 1%, more preferably at least about 5%, even more preferably at least about 15%.

The term “particle size” as used herein refers to the average mean particle size of a group of particles as seen in a composition of the present invention using a Zeiss Akzioskop at 400× magnification.

Viscosity measurements are achieved using a Brookfield R/S Rheometer at a shear rate of 2 s⁻¹ for 3 minutes.

A. Water-Soluble Detersive Surfactant

The composition of the present invention includes a water-soluble detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%.

Preferred anionic surfactants suitable for use in the compositions are the alkyl and alkyl ether sulfates. These materials have the respective formulae ROSO₃M and RO(C₂H₄O)_{X}SO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Preferably, R has from about 8 to about 18 carbon atoms, more preferably from about 10 to about 16 carbon atoms, even more preferably from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with from about 0 and about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula R¹—SO₃-M wherein R¹ is a straight or branched chain, saturated, aliphatic hydrocarbon radical having from about 8 to about 24, preferably from about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having from about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. No. 3,332,880.

Another class of anionic detersive surfactants suitable for use in the compositions is the beta-alkyloxy alkane sulfonates. These surfactants conform to the formula: 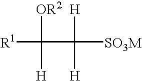
where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof.

Suitable zwitterionic or amphoteric detersive surfactants for use in the composition herein include those which are known for use in hair care or other personal cleansing compositions. Concentration of such amphoteric detersive surfactants preferably ranges from about 0.5% to about 20%, preferably from about 1% to about 10%. Non-limiting examples of suitable zwitterionic or amphoteric surfactants are described in U.S. Pat. Nos. 5,104,646 and 5,106,609, both to Bolich Jr. et al.

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines are preferred.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678; 2,658,072; 2,438,091; and 2,528,378.

B. Fibers

The personal cleansing compositions of the present invention comprise a fiber. As used herein, a “fiber” is a natural or synthetic object which is slender and elongated in shape, having a length and a thickness such that the length is greater than the thickness, and having a cross-section which is substantially circular, substantially elliptical, or substantially polygonal. The term “fiber” includes substances which are, for example, rod-like or ribbon-like in shape.

The fibers of the present invention form a load-sensitive deposit upon dilution of the personal cleansing composition with water. As used herein, a “load-sensitive deposit” is a material which, at very low force applied to the hair, acts to increase hair friction relative to the hair friction of clean hair, and, at higher force applied to the hair, acts to decrease hair friction relative to the hair friction at very low force. In one embodiment of the present invention, the load-sensitive deposit forms upon dilution of the personal cleansing composition with water at a ratio of water to personal cleansing composition of at least about 1:1.

To deliver positive hair volume and body effects through the use of a deposit onto the hair while still maintaining positive conditioning and combing performance, the deposit must perform differently under rest conditions and under combing conditions. Because the amount of force used to comb hair is magnitudes greater than the amount of force that hairs exert on other hairs when the hair is at rest, a deposit on the hair which provides a hair friction profile which changes as force applied to the hair changes can provide improved hair volume, body, and fullness, while maintaining a good conditioning and combing performance.

When deposited on hair, the fibers of the present invention provide a hair friction profile which changes as the force applied to the hair changes. Specifically, at very low force applied to the hair, the fibers of the present invention become physically oriented on the hair in such a way that hair friction increases relative to the friction of clean hair, preventing hairs from moving past other hairs. At higher force applied to the hair, hair friction decreases, allowing positive conditioning and combing performance.

It is believed that the fibers of the present invention provide a decrease in friction at higher force applied to the hair because of the bending modulus of the fiber. Fibers having a higher bending modulus (i.e., fibers which require more force to bend) will provide less of a decrease in hair friction at higher loads compared to fibers having a lower bending modulus. Therefore, through selection of fibers of different material and size, the friction profile can be controlled and altered.

The fibers of the present invention have a length from about 5 μm to about 1.5 mm, preferably from about 10 μm to about 1 mm, more preferably from about 10 μm to about 500 μm.

The fibers of the present invention have a thickness from about 5 μm to about 500 μm, preferably from about 5 μm to about 80 μm.

The length of the fiber and the thickness of the fiber are selected such that the length is greater than the thickness. Preferably, the fibers of the present invention have a length and a thickness such that the aspect ratio is greater than about 5, more preferably greater than about 10, even more preferably greater than about 20. As used herein, the term “aspect ratio” refers to the ratio of the largest dimension of an object to the smallest dimension of an object.

The fibers of the present invention may be natural or synthetic, inorganic or organic in origin. Examples of suitable fibers include fibers of silk, cotton, wool, flax, cellulose extracts, wood, vegetables or algae, polyamide (Nylon®), modified cellulose (rayon, viscose, acetate, especially rayon acetate), poly-p-phenylene terephthalamide, Kevlar®, acrylic, methyl polymethacrylate or poly 2-hydroxyethyl methacrylate, polyolefin and polyethylene or polypropylene, glass, silica, aramid, carbon (especially in the form of graphite), Teflon®, insoluble collagen, polyesters, poly vinyl or vinylidene chloride, polyvinyl alcohol, polyacrylonitrile, chitosan, polyurethane, polyethylene phthalate, fibers formed of a mixture of polymers such as those mentioned above such as fibers of polyamide/polyester, and mixtures thereof.

Preferred fibers of the present invention include cellulose, polyamide (Nylon®), polyethylene, silica, Teflon®, polyurethane, polypropylene, titanium dioxide, and mixtures thereof.

In one embodiment of the present invention, the fibers are cellulosic fibers having a length from about 5 μm to about 500 μm and a thickness from about 5 μm to about 80 μm. These cellulosic fibers are ribbon-like in shape. Such cellulosic fibers are available, for example, from Kobo Products, Inc., under the trade name Cell-U-Lash.

In another embodiment of the present invention, the fibers are polyamide fibers having a length from about 0.9 mm to about 1.2 mm and a thickness from about 14 μm to about 20 μm. These polyamide fibers are rod-like in shape, having a substantially circular cross-section. Such polyamide fibers are available, for example, from Kobo Products, Inc., under the trade name Nylon Cut Fiber.

The personal cleansing compositions of the present invention comprise at least about 0.01 wt. % of a fiber, preferably from about 0.2 wt. % to about 5 wt. %.

C. Aqueous Carrier

The compositions of the present invention are typically in the form of pourable liquids (under ambient conditions), but may also be in the form of gels, lotions, creams, mousses, and sprays. The compositions, therefore, will comprise an aqueous carrier, which is present at a level of from about 20% to about 95% by weight of the composition. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

D. Additional Components

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10%.

Non-limiting examples of optional components for use in the composition include cationic polymers, particles, conditioning agents (e.g., silicones, hydrocarbon oils, fatty esters), anti-dandruff agents, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

1. Cationic Polymers

The compositions of the present invention may contain a cationic polymer to aid in deposition of the fibers and enhance conditioning performance. Suitable cationic polymers have a cationic charge density from about 1.2 meq/g to about 7.0 meq/g, preferably from about 1.5 meq/g to about 3.0 meq/g, more preferably from about 1.7 meq/g to about 2.5 meq/g, at the pH of intended use of the shampoo composition, which pH will generally range from about pH 3 to about pH 9, preferably from about pH 4 to about pH 8. The pH of the compositions of the present invention are measured neat. The average molecular weight of such suitable cationic polymers is between about 10,000 and about 10 million, preferably between about 50,000 and about 5 million, more preferably between about 100,000 and about 3 million.

Such cationic polymers may be present in the composition from about 0.01% to about 3%, preferably from about 0.05% to about 2.0%, more preferably from about 0.1% to about 1.0%, by weight of the composition.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non-limiting examples of such counterions include halides (e.g., chloride, fluoride, bromide, iodide), sulfate and methylsulfate.

Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium 6 and Polyquaternium 7, respectively); amphoteric copolymers of acrylic acid including copolymers of acrylic acid and dimethyldiallylammonium chloride (Polyquaternium 22), terpolymers of acrylic acid with dimethyldiallylammonium chloride and acrylamide (Polyquaternium 39), and terpolymers of acrylic acid with methacrylamidopropyl trimethylammonium chloride and methylacrylate (Polyquaternium 47). Preferred cationic substituted monomers are the cationic substituted dialkylaminoalkyl acrylamides, dialkylaminoalkyl methacrylamides, and combinations thereof.

Other suitable cationic polymers include those which conform to the formula: 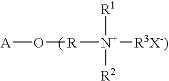
wherein A is an anhydroglucose residual group, such as a starch or cellulose anhydroglucose residual; R is an alkylene oxyalkylene, polyoxyalkylene, or hydroxyalkylene group, or combination thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers, such as Polymer KG30M having an average charge density of 1.9 meq/g and a molecular weigh of 1.5-2.0 million. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially avaialable from Rhone-Poulenc Incorporated and the N-Hance series commercially available from Aqualon Division of Hercules, Inc.

When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

2. Particles

The composition of the present invention may additionally include particles. Suitable particles have a particle size of less than about 100 μm and are present in the composition from about 0.05% to about 20%, preferably less than about 10%, more preferably less than about 5%, by weight of the composition.

The type of particle and the amount it is present are selected for the particular purpose of the composition. For example, where it is desired to deliver color benefits, pigment particles conferring the desired hues can be incorporated. Determination of the levels and particle types is within the skill of the artisan. Particles that are generally recognized as safe, and are listed in C.T.F.A. Cosmetic Ingredient Handbook, Sixth Ed., Cosmetic and Fragrance Assn., Inc., Washington D.C. (1995), incorporated herein by reference, can be used.

Suitable particles include, for example, silica, polymethylmethacrylate, acrylate polymers, aluminum silicate, aluminum starch octenylsuccinate, cellulose, hydrated silica, microcrystaline cellulose, titanium dioxide, polyethylene, alumina, calcium carbonate, nylon, silicone resins, polypropylene, polytetrafluoroethylene, polyurethane, polyamide, epoxy resins and mixtures thereof. The above mentioned particles may be surface treated with lecithin, amino acids, mineral oil, silicone oil, or various other agents either alone or in combination, which coat the particles surface and render them hydrophobic in nature.

Preferred particles include hydrophillic and hydrophobically modified precipitated silicas and aluminas, polyethylene, silicone resins and mixtures thereof.

3. Conditioning Agents

Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present invention typically comprise a water-insoluble, water-dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of the conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

a. Silicone Conditioning Agents

The conditioning agent of the compositions of the present invention is preferably a water-insoluble silicone conditioning agent. The silicone conditioning agent may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The silicone conditioning agent may be present from about 0.01% to about 10%, preferably from about 0.1% to about 5%, more preferably from about 0.2% to about 3%, by weight of the composition. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

In an opaque composition embodiment of the present invention, the personal care composition comprises a non-volatile silicone oil having a particle size as measured in the personal care composition from about 1 μm to about 50 μm. In an embodiment of the present invention for small particle application to the hair, the personal care composition comprises a non-volatile silicone oil having a particle size as measured in the personal care composition from about 100 nm to about 1 μm. A substantially clear composition embodiment of the present invention comprises a non-volatile silicone oil having a particle size as measured in the personal care composition of less than about 100 nm.

Non-volatile silicone oils suitable for use in compositions of the present invention may be selected from organo-modified silicones and fluoro-modified silicones. In one embodiment of the present invention, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

b. Organic Conditioning Oils

The compositions of the present invention may also comprise at least one organic conditioning oil as the conditioning agent, either alone or in combination with other conditioning agents, such as the silicones described above. Such organic conditioning oils are present from about 0.05% to about 3%, preferably from about 0.08% to about 1.5%, more preferably from about 0.1% to about 1%, by weight of the composition.

i. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation.

ii. Polyolefins

Organic conditioning oils for use in the compositions of the present invention can also include liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

iii. Fatty Esters

Other suitable organic conditioning oils for use as the conditioning agent in the compositions of the present invention include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to, iso-propyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are those known as polyhydric alcohol esters. Such polyhydric alcohol esters include alkylene glycol esters.

Still other fatty esters suitable for use in the compositions of the present invention are glycerides, including, but not limited to, mono-, di-, and tri-glycerides, preferably di- and tri-glycerides, more preferably triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

c. Other Conditioning Agents

i. Quaternary Ammonium Compounds

Suitable quaternary ammonium compounds for use as conditioning agents in the personal care compositions of the present invention include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in a composition of the present invention include, but are not limited to, Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

ii. Polyalkylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M, and mixtures thereof.

4. Anti-Dandruff Agents

The compositions of the present invention may also contain an anti-dandruff active. Suitable non-limiting examples of anti-dandruff actives include pyridinethione salts, azoles, selenium sulfide, particulate sulfur, and mixtures thereof. Preferred are pyridinethione salts. Such anti-dandruff particulate should be physically and chemically compatible with the essential components of the composition, and should not otherwise unduly impair product stability, aesthetics or performance.

The compositions of the present invention may further include one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulfide, sodium thiosulfate, keratolytic agents such as salicylic acid, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, extracts of melaleuca, charcoal, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof.

When present in the composition, the anti-dandruff active is included in an amount from about 0.01% to about 5%, preferably from about 0.1% to about 3%, and more preferably from about 0.3% to about 2%, by weight of the composition.

5. Suspending Agents

The compositions of the present invention may further comprise a suspending agent at concentrations effective for suspending water-insoluble material in dispersed form in the compositions or for modifying the viscosity of the composition. Such concentrations generally range from about 0.1% to about 10%, preferably from about 0.3% to about 5.0%, by weight of the composition, of suspending agent.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other optional suspending agents include crystalline suspending agents which can be categorized as acyl derivatives, long chain amine oxides, and mixtures thereof. These suspending agents include ethylene glycol esters of fatty acids preferably having from about 16 to about 22 carbon atoms. Other suitable suspending agents include alkanol α-amides of fatty acids, preferably having from about 16 to about 22 carbon atoms, more preferably about 16 to about 18 carbon atoms. Other long chain acyl derivatives include long chain esters of long chain fatty acids, long chain esters of long chain alkanol amides, and glyceryl esters.

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, and secondary amines having two fatty alkyl moieties each having at least about 12 carbon atoms. Still other suitable suspending agents include di(hydrogenated tallow)phthalic acid amide, and crosslinked maleic anhydride-methyl vinyl ether copolymer.

6. Paraffinic Hydrocarbons

The compositions of the present invention may contain one or more paraffinic hydrocarbons. Paraffinic hydrocarbons suitable for use in compositions of the present invention include those materials which are known for use in hair care or other personal care compositions, such as those having a vapor pressure at 1 atm of equal to or greater than about 21° C. (about 70° F.). Non-limiting examples include pentane and isopentane.

7. Propellants

The composition of the present invention also may contain one or more propellants. Propellants suitable for use in compositions of the present invention include those materials which are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

8. Other Optional Components

The compositions of the present invention may contain fragrance.

The compositions of the present invention may also contain water-soluble and water-insoluble vitamins such as vitamins B1, B2, B6, B12, C, pantothenic acid, pantothenyl ethyl ether, panthenol, biotin, and their derivatives, and vitamins A, D, E, and their derivatives. The compositions of the present invention may also contain water-soluble and water-insoluble amino acids such as asparagine, alanine, indole, glutamic acid, and their salts, and tyrosine, tryptamine, lysine, histadine, and their salts.

The compositions of the present invention may also contain chelating agents.

E. Method of Making

The compositions of the present invention, in general, may be made by mixing the ingredients together at either room temperature or at elevated temperature, e.g., about 72° C. Heat only needs to be used if solid ingredients are in the composition. The ingredients are mixed at the batch processing temperature. Additional ingredients, including electrolytes, polymers, and particles, may be added to the product at room temperature.

F. Method of Use

The personal cleansing compositions of the present invention are used in a conventional manner for providing both increased volume and superior styling and conditioning to hair. An effective amount of the composition for providing both increased volume and superior styling and conditioning to hair is applied to the hair, which has preferably been wetted with water, and then rinsed off. Such effective amounts generally range from about 1 g to about 50 g, preferably from about 1 g to about 20 g. Application to the hair typically includes working the composition through the hair such that most or all of the hair is contacted with the composition.

This method for providing both increased volume and superior styling and conditioning to hair comprises the steps of: (a) wetting the hair with water; (b) applying an effective amount of the personal cleansing composition to the hair; and (c) rinsing the applied areas of hair with water. These steps can be repeated as many times as desired to achieve the desired cleansing and conditioning benefit.

The personal care compositions of this invention may be used as liquids, solids, semi-solids, flakes, gels, placed in a pressurized container with a propellant added, or used in a pump spray form. The viscosity of the product may be selected to accommodate the form desired.

### NON-LIMITING EXAMPLES

The compositions illustrated in the following Examples illustrate specific embodiments of the compositions of the present invention, but are not intended to be limiting thereof. Other modifications can be undertaken by the skilled artisan without departing from the spirit and scope of this invention. These exemplified embodiments of the composition of the present invention provide cleansing of hair and volumizing benefits with good wet conditioning and combing performance.

The compositions illustrated in the following Examples are prepared by conventional formulation and mixing methods, an example of which is set forth herein below. All exemplified amounts are listed as weight percents and exclude minor materials such as diluents, preservatives, color solutions, imagery ingredients, botanicals, and so forth, unless otherwise specified.

The compositions of the present invention may be prepared using conventional formulation and mixing techniques. Where melting or dissolution of solid surfactants or wax components is required these can be added to a premix of the surfactants, or some portion of the surfactants, mixed and heated to melt the solid components, e.g., about 72° C. This mixture can then optionally be processed through a high shear mill and cooled, and then the remaining components are mixed in. The compositions of the present invention, prior to the addition of materials such as gellants or propellants, typically have a viscosity of from about 2,000 cps to about 20,000 cps. The viscosity of the composition can be adjusted by conventional techniques including addition of sodium chloride or ammonium xylenesulfonate as needed. The listed formulations, therefore, comprise the listed components and any minor materials associated with such components.

The following are representative of shampoo compositions of the invention:

| | Examples | | | | | |
|---|---|---|---|---|---|---|
| Ingredient | 1 | 2 | 3 | 4 | 5 | 6 |
| Sodium Laureth-3 Sulfate | 10 | 14 | 14 | 14 | 10 | 10 |
| Sodium Lauryl Sulfate | 6 | 2 | 2 | 2 | 6 | 6 |
| Ammonium Laureth-3 Sulfate | — | — | — | — | — | — |
| Ammonium Lauryl Sulfate | — | — | — | — | — | — |
| Cocamidopropylbetaine | — | — | — | — | — | — |
| Polyquat 10 (1) | 0.25 | — | — | — | 0.25 | 0.15 |
| Polyquat 10 (2) | — | 0.25 | — | — | — | — |
| Polyquat 10 (3) | — | — | 0.25 | — | — | — |
| Polyquat 10 (4) | — | — | — | 0.5 | — | — |
| Cellulose Fiber (5) | — | 1 | — | — | — | — |
| Cellulose Fiber (6) | — | — | — | 1 | — | — |
| Nylon Fiber (7) | — | — | 1 | — | — | — |
| Nylon Fiber (8) | 1 | — | — | — | — | — |
| Polypropylene Fiber (9) | — | — | — | — | 1 | — |
| Nylon Fiber (10) | — | — | — | — | — | 1 |
| Cellulose Fiber (11) | — | — | — | — | — | — |
| Cellulose Fiber (12) | — | — | — | — | — | — |
| Cellulose Fiber (13) | — | — | — | — | — | — |
| Dimethicone (14) | — | — | — | — | — | — |
| Dimethicone (15) | — | — | — | — | — | — |
| Polybutene (16) | — | — | — | — | — | — |
| Polybutene (17) | — | — | — | — | — | — |
| hydrophobic precipitated | — | — | — | — | — | — |
| silica (18) | | | | | | |
| precipitated silica (19) | — | — | — | — | — | — |
| polymethylsilsesquioxane (20) | — | — | — | — | — | — |
| trihydroxystearin (21) | 0.5 | 0.25 | 0.25 | 0.15 | 0.25 | 0.1 |
| Cocamide MEA | 0.8 | 0.8 | 0.8 | 0.8 | 0.8 | 0.8 |
| Perfume Solution | 0.7 | 0.7 | 0.7 | 0.7 | 0.7 | 0.7 |
| Citric Acid | 0.23 | 0.23 | 0.23 | 0.23 | 0.23 | 0.23 |
| Sodium Benzoate | 0.25 | 0.25 | 0.25 | 0.25 | 0.25 | 0.25 |
| Sodium Chloride | 1.5 | 2 | 1 | 2 | 1.5 | 1.5 |
| Water and Minors | q.s. | q.s. | q.s. | q.s. | q.s. | q.s. |
| (q.s. to 100%) | | | | | | |

(1) Polymer KG30M available from Amerchol
(2) Polymer JP available from Amerchol
(3) Polymer KG-4M available from Amerchol
(4) Polymer JR-30M available form Amerchol
(5) Cell-U-Lash 150 available from Kobo Products Inc.
(6) Cell-U-Lash 40 available from Kobo Products Inc.
(7) Nylon Cut Fiber 1.5D available from Kobo Products Inc.
(8) Nylon Cut Fiber 3D available from Kobo Products Inc.
(9) PP Fiber 6D05 available from Kobo Products Inc.
(10) Nylon Cut Fiber 3D available from Kobo Products Inc.
(11) CreaFibe SC 40 from CreaFill Fibers Corp.
(12) CreaFibe SC 150G from CreaFill Fibers Corp.
(13) CreaFibe SC 200 from CreaFill Fibers Corp.
(14) Viscasil 330M available from General Electric Silicones
(15) Emulsion of 70,000 csk polydimethylsiloxane with a particle size of approximately 30 nm available as DC1870 from Dow Corning
(16) Indopol H50 available from BP
(17) Indopol 1900 available from BP
(18) Sipernat D11 available from Degussa
(19) Sipernat 22LS available from Degussa
(20) Tospearl 3120 available from GE Silicones
(21) Thixin R available from Rheox, Inc.

| | Examples | | | | | |
|---|---|---|---|---|---|---|
| Ingredient | 7 | 8 | 9 | 10 | 11 | 12 |
| Sodium Laureth-3 Sulfate | 10 | 10 | 10 | 10 | 10 | 10 |
| Sodium Lauryl Sulfate | 6 | 6 | 6 | 6 | 6 | 6 |
| Ammonium Laureth-3 Sulfate | — | — | — | — | — | — |
| Ammonium Lauryl Sulfate | — | — | — | — | — | — |
| Cocamidopropylbetaine | — | — | — | — | — | — |
| Polyquat 10 (1) | 0.25 | 0.5 | 0.25 | 0.5 | 0.25 | 0.25 |
| Polyquat 10 (2) | — | — | — | — | — | — |
| Polyquat 10 (3) | — | — | — | — | — | — |
| Polyquat 10 (4) | — | — | — | — | — | — |
| Cellulose Fiber (5) | — | — | — | 0.5 | 0.5 | — |
| Cellulose Fiber (6) | — | — | — | — | — | 0.25 |
| Nylon Fiber (7) | — | — | — | 0.5 | — | — |
| Nylon Fiber (8) | — | — | — | — | — | — |
| Polypropylene Fiber (9) | — | — | — | — | — | — |
| Nylon Fiber (10) | — | — | — | — | — | — |
| Cellulose Fiber (11) | 0.75 | — | — | — | — | — |
| Cellulose Fiber (12) | — | 1 | — | — | — | — |
| Cellulose Fiber (13) | — | — | 1 | — | — | — |
| Dimethicone (14) | — | — | — | — | — | — |
| Dimethicone (15) | — | — | — | 0.25 | — | — |
| Polybutene (16) | — | — | — | — | — | — |
| Polybutene (17) | — | — | — | — | — | — |
| hydrophobic precipitated | — | — | — | — | 1 | — |
| silica (18) | | | | | | |
| precipitated silica (19) | — | — | — | — | — | 0.25 |
| polymethylsilsesquioxane (20) | — | — | — | — | — | — |
| trihydroxystearin (21) | 0.25 | 0.5 | 0.15 | 0.5 | 0.25 | 0.25 |
| Cocamide MEA | 0.8 | 0.8 | 0.8 | 0.8 | 0.8 | 0.8 |
| Perfume Solution | 0.7 | 0.7 | 0.7 | 0.7 | 0.7 | 0.7 |
| Citric Acid | 0.23 | 0.23 | 0.23 | 0.23 | 0.23 | 0.23 |
| Sodium Benzoate | 0.25 | 0.25 | 0.25 | 0.25 | 0.25 | 0.25 |
| Sodium Chloride | 1.5 | 1.5 | 1.5 | 1.5 | 1.5 | 1.5 |
| Water and Minors | q.s. | q.s. | q.s. | q.s. | q.s. | q.s. |
| (q.s. to 100%) | | | | | | |

(1) Polymer KG30M available from Amerchol
(2) Polymer JP available from Amerchol
(3) Polymer KG-4M available from Amerchol
(4) Polymer JR-30M available form Amerchol
(5) Cell-U-Lash 150 available from Kobo Products Inc.
(6) Cell-U-Lash 40 available from Kobo Products Inc.
(7) Nylon Cut Fiber 1.5D available from Kobo Products Inc.
(8) Nylon Cut Fiber 3D available from Kobo Products Inc.
(9) PP Fiber 6D05 available from Kobo Products Inc.
(10) Nylon Cut Fiber 3D available from Kobo Products Inc.
(11) CreaFibe SC 40 from CreaFill Fibers Corp.
(12) CreaFibe SC 150G from CreaFill Fibers Corp.
(13) CreaFibe SC 200 from CreaFill Fibers Corp.
(14) Viscasil 330M available from General Electric Silicones
(15) Emulsion of 70,000 csk polydimethylsiloxane with a particle size of approximately 30 nm available as DC1870 from Dow Corning
(16) Indopol H50 available from BP
(17) Indopol 1900 available from BP
(18) Sipernat D11 available from Degussa
(19) Sipernat 22LS available from Degussa
(20) Tospearl 3120 available from GE Silicones
(21) Thixin R available from Rheox, Inc.

| | Examples | | | | | |
|---|---|---|---|---|---|---|
| Ingredient | 13 | 14 | 15 | 16 | 17 | 18 |
| Sodium Laureth-3 Sulfate | 10 | 8 | 8 | — | — | — |
| Sodium Lauryl Sulfate | 6 | 8 | 4 | — | — | — |
| Ammonium Laureth-3 Sulfate | — | — | — | 10 | 12.5 | 8 |
| Ammonium Lauryl Sulfate | — | — | — | 6 | 1.5 | 8 |
| Cocamidopropylbetaine | — | — | 4 | — | 2.7 | — |
| Polyquat 10 (1) | 0.25 | 0.25 | 0.5 | 0.25 | 0.5 | — |
| Polyquat 10 (2) | — | — | — | — | — | — |
| Polyquat 10 (3) | — | — | — | — | — | 0.75 |
| Polyquat 10 (4) | — | — | — | — | — | — |
| Cellulose Fiber (5) | 1 | 2 | — | 1 | — | — |
| Cellulose Fiber (6) | — | — | — | — | — | — |
| Nylon Fiber (7) | — | — | — | — | 2 | — |
| Nylon Fiber (8) | — | — | — | — | — | — |
| Polypropylene Fiber (9) | — | — | — | — | — | — |
| Nylon Fiber (10) | — | — | — | — | — | 1 |
| Cellulose Fiber (11) | — | — | 1 | — | — | — |
| Cellulose Fiber (12) | — | — | — | — | — | — |
| Cellulose Fiber (13) | — | — | — | — | — | — |
| Dimethicone (14) | — | — | — | 1 | — | — |
| Dimethicone (15) | — | — | — | — | 1 | — |
| Polybutene (16) | — | — | — | 1 | — | 1 |
| Polybutene (17) | — | — | — | — | 0.5 | — |
| hydrophobic precipitated | — | — | — | — | — | — |
| silica (18) | | | | | | |
| precipitated silica (19) | — | — | — | — | — | — |
| polymethylsilsesquioxane (20) | 2 | — | — | — | — | — |
| trihydroxystearin (21) | 0.3 | 0.3 | 0.1 | 0.5 | 0.5 | 0.25 |
| Cocamide MEA | 0.8 | 0.8 | 0.8 | 0.8 | 0.8 | 0.8 |
| Perfume Solution | 0.7 | 0.7 | 0.7 | 0.55 | 0.55 | 0.55 |
| Citric Acid | 0.23 | 0.23 | 0.23 | 0.04 | 0.04 | 0.04 |
| Sodium Benzoate | 0.25 | 0.25 | 0.25 | 0.25 | 0.25 | 0.25 |
| Sodium Chloride | 1.5 | 0.2 | 1.5 | 0.5 | 3.5 | 0 |
| Water and Minors | q.s. | q.s. | q.s. | q.s. | q.s. | q.s. |
| (q.s. to 100%) | | | | | | |

(1) Polymer KG30M available from Amerchol
(2) Polymer JP available from Amerchol
(3) Polymer KG-4M available from Amerchol
(4) Polymer JR-30M available form Amerchol
(5) Cell-U-Lash 150 available from Kobo Products Inc.
(6) Cell-U-Lash 40 available from Kobo Products Inc.
(7) Nylon Cut Fiber 1.5D available from Kobo Products Inc.
(8) Nylon Cut Fiber 3D available from Kobo Products Inc.
(9) PP Fiber 6D05 available from Kobo Products Inc.
(10) Nylon Cut Fiber 3D available from Kobo Products Inc.
(11) CreaFibe SC 40 from CreaFill Fibers Corp.
(12) CreaFibe SC 150G from CreaFill Fibers Corp.
(13) CreaFibe SC 200 from CreaFill Fibers Corp.
(14) Viscasil 330M available from General Electric Silicones
(15) Emulsion of 70,000 csk polydimethylsiloxane with a particle size of approximately 30 nm available as DC1870 from Dow Corning
(16) Indopol H50 available from BP
(17) Indopol 1900 available from BP
(18) Sipernat D11 available from Degussa
(19) Sipernat 22LS available from Degussa
(20) Tospearl 3120 available from GE Silicones
(21) Thixin R available from Rheox, Inc.

The following are representative of conditioner compositions of the present invention:

| | Examples | | | |
|---|---|---|---|---|
| Ingredient | 19 | 20 | 21 | 22 |
| L-Glutamic Acid | 0.640 | 0.412 | — | — |
| Stearamidopropyldimethylamine | 2.000 | 1.600 | 1.000 | |
| Behentrimonium Chloride | — | — | — | 3.380 |
| Quaterium-18 | — | — | 0.750 | — |
| Cetyl Alcohol | 2.500 | 2.000 | 0.960 | 2.320 |
| Stearyl Alcohol | 4.500 | 3.600 | 0.640 | 4.180 |
| Cetearyl Alcohol | — | — | 0.500 | — |
| Polysorbate 60 | — | — | 0.500 | — |
| Glyceral Monostearate | — | — | 0.250 | — |
| Oleyl Alcohol | — | — | 0.250 | — |
| Hydroxyethylcellulose | — | — | 0.250 | — |
| Peg 2M (1) | — | — | 0.500 | — |
| Dimethicone (2) | — | 0.200 | — | — |
| Dimethicone (3) | 0.630 | — | 0.630 | 0.630 |
| Cyclopentasiloxane (3) | 3.570 | — | 3.570 | 3.570 |
| Benzyl Alcohol | 0.400 | 0.400 | 0.400 | 0.400 |
| Methyl Paraben | 0.200 | 0.200 | 0.200 | 0.200 |
| Propyl Paraben | 0.100 | 0.100 | 0.100 | 0.100 |
| Phenoxy Ethanol | 0.300 | 0.300 | 0.300 | 0.300 |
| Sodium Chloride | 0.010 | 0.010 | — | — |
| Citric Acid | 0.130 | 0.130 | 0.200 | — |
| Kathon | — | — | — | — |
| Perfume | 0.400 | 0.400 | 0.400 | 0.300 |
| Sodium Hydroxide | — | — | — | 0.014 |
| Isopropyl Alcohol | — | — | — | 0.507 |
| Cellulose Fiber (4) | 1.000 | — | — | 0.500 |
| Nylon Fiber (5) | — | 1.000 | — | 0.500 |
| Polypropylene Fiber (6) | — | — | 1.000 | — |
| Water and Minors (q.s. to 100%) | q.s. | q.s. | q.s. | q.s. |

(1) Polyox WSR N-10 available from Amerchol Corp.
(2) 10,000 cps Dimethicone TSF451-1MA available from GE
(3) 15/85 Dimethicone/Cyclomethicone Blend available from GE
(4) Cell-U-Lash 150 available from Kobo Products Inc.
(5) Nylon Cut Fiber 1.5D available from Kobo Products Inc.
(6) PP Fiber 6D05 available from Kobo Products Inc.

All documents cited herein are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A personal cleansing composition comprising:
a) from about 5 wt. % to about 50 wt. % of a water-soluble detersive surfactant;
b) at least about 0.1 wt. % of a fiber having a length and a thickness such that said length is greater than said thickness, wherein said length is from about 5 μm to about 1.5 mm; and
c) at least about 20 wt. % of an aqueous carrier;
wherein said fiber forms a load-sensitive deposit upon dilution of said personal cleansing composition with water.

2. A personal cleansing composition according to claim 1, comprising from about 0.2 wt. % to about 5 wt. % of said fiber.

3. A personal cleansing composition according to claim 1, wherein said thickness is from about 5 μm to about 500 μm.

4. A personal cleansing composition according to claim 1, wherein said fiber has a length from about 10 μm to about 500 μm.

5. A personal cleansing composition according to claim 1, wherein said fiber is selected from the group consisting of cellulose fibers, polyamide fibers, polyethylene fibers, silica fibers, polytetrafluoroethylene fibers, polyurethane fibers, polypropylene fibers, titanium dioxide fibers, and mixtures thereof.

6. A personal cleansing composition according to claim 1, wherein said load-sensitive deposit forms upon dilution of said personal cleansing composition with water at a ratio of water to personal cleansing composition of at least about 1:1.

7. A personal cleansing composition according to claim 1, further comprising a cationic polymer.

8. A personal cleansing composition according to claim 7, wherein said cationic polymer has a charge density from about 1.2 meq/g to about 7.0 meq/g and a molecular weight from about 10,000 to about 10,000,000.

9. A personal cleansing composition according to claim 8, wherein said cationic polymer has a charge density from about 1.5 meq/g to about 3.0 meq/g.

10. A personal cleansing composition according to claim 8, wherein said cationic polymer has a charge density from about 1.7 meq/g to about 2.5 meq/g.

11. A personal cleansing composition according to claim 7, wherein said cationic polymer is selected from the group consisting of cationic cellulose derivative and cationic guar gum derivative.

12. A personal cleansing composition according to claim 1, further comprising a particle selected from the group consisting of silica, hydrated silica, polymethylmethacrylate, acrylate polymers, aluminum silicate, aluminum starch octenylsuccinate, cellulose, microcrystaline cellulose, titanium dioxide, polyethylene, alumina, calcium carbonate, nylon, silicone resins, polypropylene, polytetrafluoroethylene, polyurethane, polyamide, epoxy resins, and mixtures thereof.

13. A personal cleansing composition according to claim 1, further comprising a conditioning agent.

14. A personal cleansing composition according to claim 13, wherein said conditioning agent is selected from the group consisting of silicone conditioning agents, hydrocarbon oils, polyolefins, fatty esters, and mixtures thereof.

15. A personal cleansing composition according to claim 1, further comprising one or more additional components selected from the group consisting of anti-dandruff agents, suspending agents, paraffinic hydrocarbons, and propellants.

16. A personal cleansing composition comprising:
a) from about 5 wt. % to about 50 wt. % of a water-soluble detersive surfactant;
b) at least about 0.1 wt. % of a fiber having a length and a thickness such that said length is greater than said thickness, wherein said length is from about 5 μm to about 1.5 mm;
c) from about 0.01 wt. % to about 3 wt. % of a cationic polymer; and
d) at least about 20 wt. % of an aqueous carrier;
wherein said fiber forms a load-sensitive deposit upon dilution of said personal cleansing composition with water.

17. A method of providing both increased volume and superior styling and conditioning to hair, said method comprising the steps of:
a) applying to wet hair a composition comprising:
i) from about 5 wt. % to about 50 wt. % of a detersive surfactant;
ii) at least about 0.1 wt. % of a fiber having a length and a thickness such that said length is greater than said thickness, wherein said length is from about 5 μm to about 1.5 mm; and
iii) at least about 20 wt. % of an aqueous carrier;
wherein said fiber forms a load-sensitive deposit upon dilution of said composition with water; and
b) rinsing said composition from said hair.
